(19) United States
(12) Patent Application Publication
(10) Pub. No.: US 2026/0174479 A1
(43) Pub. Date: Jun. 25, 2026

(54) NON-INVASIVELY ADJUSTABLE BONE PROSTHESIS

(71) Applicant: Granite Medical, LLC, Lebanon, NH (US)

(72) Inventor: Jeffrey David Gordon, Hanover, NH (US)

(73) Assignee: Granite Medical, LLC, Lebanon, NH (US)

(21) Appl. No.: 18/991,293

(22) Filed: Dec. 20, 2024

(51) Int. Cl. A61B 17/74; A61F 2/28

(52) U.S. Cl. CPC A61B 17/748; A61F 2002/2825

(57) ABSTRACT

An endoprosthesis intended to replace a resected portion of bone and joint. The endoprosthesis can be non-invasively, manually operated to lengthen a patient's limb both intraoperatively and postoperatively. It includes bone anchoring components for anchoring to a first and second long bone, a joint to replace the function of an orthopedic joint, and a purely mechanical lengthening mechanism which can be located and operated manually by an operator by pressing on skin of a patient without requiring a surgical incision. The axle for joint is the actuating mechanism that lengthens the implant. The endoprosthesis does not need or include any magnets, electric motors, electric current, application of heat or a fluid displacement (hydraulic) system.

### STATEMENT REGARDING FEDERALLY SPONSORED RESEARCH OR DEVELOPMENT

This invention was made with Government support under Award #5R44AR083875, awarded by the National Institutes of Health (NIH) and the National Institute of Arthritis and Musculoskeletal and Skin Diseases (NIAMS). The Government has certain rights in the invention.

### FIELD OF THE INVENTION

The invention relates to a non-invasively adjustable bone prosthesis that does not utilize magnets, an electric motor, or hydraulics.

### BACKGROUND OF THE INVENTION

Limb salvage surgery with a prosthesis replaces a damaged section of bone and reconstructs a functional limb. In the majority of these cases the patient requires resection of a joint such as the knee or hip. Osteosarcoma, Ewings sarcoma and extreme traumatic injury are examples of conditions requiring limb salvage. If the patient is a child or adolescent the operative limb will need to be gradually lengthened to maintain leg length equality throughout the growing years. Historically, multiple surgeries have been required to periodically lengthen the implant until the patient reaches skeletal maturity. However, the incidence of infection increases 5% with each successive surgery so that, for example, a patient requiring 10 lengthening procedures will have approximately 50% greater risk of infection compared to a patient not requiring lengthening surgery.

Magnetically actuated implants (U.S. Pat. No. 6,849,076), where an external electromagnet spins an internal permanent magnet to drive lengthening, have been developed to eliminate the need for lengthening surgery. These implants have reduced infections and have demonstrated some clinical success, but they have disadvantages. The transmission of torque to the implant from an external electromagnet is a difficult engineering challenge. The magnet located inside the implant is small and the electromagnet located outside the patient is relatively far from the implant. Magnetic attraction decreases exponentially with increasing distance between magnets, so only a small torque is generated on the implanted magnet requiring a complex miniaturized two-phase planetary gear reduction mechanism to create a large enough lengthening force. The magnet inside the implant spins at 3,000 rpm for 4 minutes in order to lengthen 1 mm. These intricate implants are costly to produce, and are prone to failure. Additionally, the drive mechanism is cumbersome so the implant is long. Young patients require the most overall lengthening from an implant but cannot accommodate the length of an implant which would provide that lengthening. Therefore, staged revision surgeries are often required where shorter implants with shorter lengthening capacity are exchanged prior to skeletal maturity. Also, magnetic implants preclude MRI imaging, a particularly useful tool for oncological assessment.

Motorized extendable implants (U.S. Pat. No. 5,961,553) have been proposed wherein a small electric motor, powered by a battery or an inductive electromagnetic source, is used to turn a lead screw to lengthen the implant. Again, these are expensive and complicated implants that risk both mechanical failure and the inability to generate the force required for lengthening.

Hydraulically extendable implants (U.S. Pat. No. 5,350,379) have been described wherein a fluid pump is activated by manually pushing a patient's skin, an internal electric fluid pump is controlled by an external system, or an external fluid pump is connected percutaneously to the implant. Hydraulic seals are prone to failure and the overall device can be imprecise. Percutaneous systems significantly increase the risk of infection.

A more simplified, robust solution is needed which does not rely on magnets, an electric motor, or hydraulics.

### SUMMARY OF THE INVENTION

The invention is made from traditional implantable materials; no magnetic materials are necessary. The invention is purely mechanical and operable with non-invasive, direct, manual manipulation of the implant by pressing on the patient's skin. No motors and no electromagnetic or hydraulic systems are needed. In the case of a distal femur replacement, the most common requirement for osteosarcoma patients, manual manipulation occurs at the medial and lateral surfaces of the knee joint at the site where the femoral condyles were located prior to resection. Implantation of a distal femur replacement typically requires an incision along the axis of the femur in the coronal plane and the skin at the medial and lateral aspects of the knee is left intact. Therefore, manual manipulation of the implant should not interfere with wound healing or cause pain at the site of the surgical incision.

While distal femur replacement and total femur replacement implants are presented here, it will be obvious to those familiar with endoprostheses that this invention could be adapted for the proximal tibia and distal humerus. In addition to cases of osteosarcoma resection, the implant presented here can be utilized for any condition requiring bone resection and replacement with a prosthesis, and subsequent lengthening—such as traumatic injuries in growing patients, injuries to the growth plate, congenital or developmental deformities, and revision arthroplasty procedures where soft tissue contracture requires gradual implant lengthening to regain limb length equality.

One aspect of the invention relates to a non-invasively extendable endoprosthesis incorporating: a first feature configured for attachment to a first long bone; a second feature configured for attachment to a second, adjacent long bone; a joint having at least one rotational degree of freedom which is intended to replace the function of an orthopedic joint; and a lengthening mechanism which is purely mechanical and manually operates while being located and activated by an operator post-operatively by pressing on skin of the patient, without requiring a surgical incision, a magnet, a hydraulic or pneumatic component, electrical current or application of heat. The lengthening mechanism has a threaded shaft rotatably connected to the first feature to lengthen or shorten the first feature when the threaded shaft is rotated; a drive ratchet engaged with the threaded shaft and having first teeth on a first side of the drive ratchet; a pinion ratchet having teeth which are engageable with the first teeth of the drive ratchet to cause rotation of the drive ratchet when the pinion ratchet is rotated in a first direction to thereby cause rotation of the threaded shaft, the teeth of the pinion ratchet disengagable from the first teeth of the drive ratchet when the pinion ratchet is rotated in a second direction opposite to the first direction; and a frame affixable to the body and supporting the drive ratchet and the pinion ratchet.

In some embodiments, the pinion ratchet and drive ratchet are mutually separable by translation to enable the pinion ratchet to disengage from the first teeth of the drive ratchet when the pinion ratchet is rotated in the second direction. The pinion ratchet rotates axially about the longitudinal axis of the threaded shaft.

The drive ratchet can include second teeth on a side of the drive ratchet opposite to the first teeth, with the endoprosthesis further including an idler ratchet translatable along the longitudinal axis of the threaded shaft and connected to the frame to limit radial rotation of the idler ratchet about the longitudinal axis of the threaded shaft. The threaded shaft is rotatable within a bore of the idler ratchet. Teeth of the idler ratchet are engageable with the second teeth of the drive ratchet to prevent rotation of the drive ratchet and thereby the threaded shaft when the drive ratchet is not being rotated in the first direction by the pinion ratchet. The idler ratchet and the drive ratchet are mutually separable from engagement by translation when the pinion ratchet is rotated in the first direction, with the drive ratchet being translatable along the longitudinal axis of the threaded shaft, and limited in radial rotation about the longitudinal axis of the threaded shaft.

A biasing element can be positioned between the frame and the idler ratchet to bias the idler ratchet, drive ratchet, and pinion ratchet into mutual engagement.

The threaded shaft can have an axially extending engagement region that is slidably engageable with a mating region within the bore of the drive ratchet to thereby prevent the radial rotation of the drive ratchet relative to the threaded shaft, while enabling translation of the drive ratchet along the longitudinal axis of the threaded shaft.

The endoprosthesis can further include an axle, the axle including a slot, the pinion ratchet including a cam, the cam of the pinion ratchet engaged within the slot, whereby movement of the axle causes relative movement of the cam causing rotation of the pinion ratchet in the first or second direction, an end of the axle pressable by the pressing on skin of the patient. The axle is disposed at an angle relative to the longitudinal axis of the threaded shaft.

In an exemplary embodiment, the frame forms a part of the joint with the axle forming a rotational axis of the joint.

Another aspect of the invention relates to an endoprosthesis extendable in length without cutting skin of the patient. The endoprosthesis comprises: a proximal end attachable to an object forming a portion of an elongate bone of the patient; a threaded shaft rotatably attached to the proximal end; a distal end connected to the threaded shaft, whereby rotation of the threaded shaft causes relative separation of the proximal and distal ends thereby lengthening the elongate bone of the patient; a drive ratchet engaged with the threaded shaft and having first teeth on a first side of the drive ratchet; a pinion ratchet having teeth which are engageable with the first teeth of the drive ratchet to cause rotation of the drive ratchet when the pinion ratchet is rotated in a first direction to thereby cause rotation of the threaded shaft, the teeth of the pinion ratchet disengagable from the first teeth of the drive ratchet when the pinion ratchet is rotated in a second direction opposite to the first direction, the pinion rachet rotatable by pressing on skin of the patient; and a frame affixable to the body and supporting the drive ratchet and the pinion ratchet. The length of the endoprosthesis is increasable by rotating the pinion ratchet by the pressing of the skin, and without a requirement of forming a surgical incision, and without using a magnet, hydraulic or pneumatic component, electrical current, or an application of heat.

In an exemplary embodiment, the frame forms a portion of a joint replacement.

In some embodiments, the pinion ratchet and drive ratchet are mutually separable by translation to enable the pinion ratchet to disengage from the first teeth of the drive ratchet when the pinion ratchet is rotated in the second direction.

In an embodiment, the pinion ratchet rotates radially about the longitudinal axis of the threaded shaft.

The drive ratchet can include second teeth on a side of the drive ratchet opposite to the first teeth, with the endoprosthesis further including an idler ratchet translatable along the longitudinal axis of the threaded shaft and connected to the frame to limit radial rotation of the idler ratchet about the longitudinal axis of the threaded shaft. The threaded shaft is rotatable within a bore of the idler ratchet. Teeth of the idler ratchet are engageable with the second teeth of the drive ratchet to prevent rotation of the drive ratchet and the threaded shaft when the drive ratchet is not being rotated in the first direction by the pinion ratchet. The drive ratchet is limited in radial rotation about the longitudinal axis of the threaded shaft, and the idler ratchet is translatable away from engagement with the drive ratchet when the pinion ratchet is rotated in the first direction.

A biasing element can be positioned between the frame and the idler ratchet to bias the idler ratchet into engagement with the drive ratchet.

In some embodiments, the threaded shaft has an axially extending engagement region that is engageable with a mating region within the bore of the drive ratchet to thereby prevent the radial rotation of the drive ratchet about the elongated axis of the threaded shaft.

The endoprosthesis can include an axle, with the axle and the pinion ratchet mutually engaged by a cam on one of the axle and pinion ratchet and a slot on the other of the axle and pinion ratchet. Movement of the axle causes relative movement of the cam and slot causing rotation of the pinion ratchet in the first or second direction, with an end of the axle pressable by the pressing on skin of the patient.

In preferred embodiments, the axle forms a rotational axis of the joint.

Another aspect of the invention relates to an endoprosthesis extendable in length without cutting skin of the patient. The endoprosthesis comprises: a proximal end attachable to an object forming a portion of an elongate bone of the patient; a threaded shaft rotatably attached to the proximal end; a distal end connected to the threaded shaft, whereby rotation of the threaded shaft causes relative separation of the proximal and distal ends thereby lengthening the elongate bone of the patient; a drive ratchet engaged with the threaded shaft and limited in radial rotation about the threaded shaft and having first teeth on a first side of the drive ratchet and second teeth on a second side opposite to the first side; a pinion ratchet having teeth which are engageable with the first teeth of the drive ratchet to cause rotation of the drive ratchet when the pinion ratchet is rotated in a first direction to thereby cause rotation of the threaded shaft, the teeth of the pinion ratchet disengagable from the first teeth of the drive ratchet by translation of the pinion ratchet relative to the drive ratchet, when the pinion ratchet is rotated in a second direction opposite to the first direction, the pinion rachet rotatable by pressing on skin of the patient; an idler ratchet connected to the frame to limit radial rotation of the idler ratchet about the longitudinal axis of the threaded shaft; the threaded shaft rotatable within a bore of the idler ratchet; teeth of the idler ratchet engageable with the second teeth of the drive ratchet to prevent rotation of the drive ratchet and thereby the threaded shaft when the drive ratchet is not being rotated in the first direction by the pinion ratchet; the drive ratchet being translatable relative to the drive pinion along the longitudinal axis of the threaded shaft to enable mutual engagement when the pinion ratchet is rotated in the first direction; a frame affixable to the body and supporting the drive ratchet and the pinion ratchet; and an axle, the axle and the pinion ratchet mutually engaged by a cam on one of the axle and pinion ratchet and a slot on the other of the axle and pinion ratchet, whereby movement of the axle causes relative movement of the cam and slot causing rotation of the pinion ratchet in the first or second direction, an end of the axle pressable by the pressing on skin of the patient. The length of the endoprosthesis is increasable by pushing the axle thereby rotating the pinion ratchet by the pressing of the skin, and without a requirement of forming a surgical incision, and without using a magnet, hydraulic or pneumatic component, electrical current, or an application of heat.

### BRIEF DESCRIPTION OF THE DRAWINGS

A more complete understanding of the present invention, and the attendant advantages and features thereof, will be more readily understood by reference to the following description when considered in conjunction with the accompanying drawings wherein:

FIG. 1 shows the preferred embodiment of the invention: a distal femur replacement;

FIG. 2 is an exploded view of the preferred embodiment of the invention;

FIG. 3 is an anterior view of the preferred embodiment of the invention;

FIG. 4 is a sectioned view of the preferred embodiment of the invention, according to the section line in FIG. 3;

FIG. 5 is a detail view of the sectioned view of the preferred embodiment of the invention shown in FIG. 4;

FIG. 6 is an exploded view of the lengthening mechanism of the preferred embodiment of the invention;

FIG. 7 shows the lengthening mechanism of the preferred embodiment of the invention under the action of a driving force F1;

FIG. 8 shows the lengthening mechanism of the preferred embodiment of the invention under the action of a driving force F2;

FIG. 9 shows the intraoperative assembly of the preferred embodiment of the invention;

FIG. 10 is an alternative embodiment of the invention: a total femur replacement;

FIG. 11 is an exploded view of a second alternative embodiment of the invention; and

FIG. 12 is a third alternative embodiment of the invention.

### DESCRIPTION OF THE EMBODIMENTS OF THE INVENTION

As required, embodiments are disclosed herein; however, it is to be understood that the disclosed embodiments are merely examples and that the devices and methods described below can be embodied in various forms. Therefore, specific structural and functional details disclosed herein are not to be interpreted as limiting, but merely as a representative basis for teaching one skilled in the art to variously employ the present subject matter in virtually any appropriately detailed structure and function. Further, the terms and phrases used herein are not intended to be limiting, but rather, to provide an understandable description of the concepts.

It can be advantageous to set forth definitions of certain words and phrases used throughout this disclosure. The terms “a” or “an”, as used herein, are employed to describe elements and components described herein. This is done merely for convenience and to give a general sense of the scope of the disclosure. This description should be read to include one or at least one and the singular also includes the plural unless it is obvious that it is meant otherwise. The term plurality, as used herein, is defined as two or more than two. The term another, as used herein, is defined as at least a second or more. The term “coupled,” as used herein, is defined as “connected,” although not necessarily directly, and not necessarily mechanically.

The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, can mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items can be used, and only one item in the list can be needed. For example, “at least one of: A, B, and C” includes any of the following combinations: A; B; C; A and B; A and C; B and C; and A, B, and C.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

Note that not all of the activities described in the general description or the examples are required, that a portion of a specific activity may not be required, and that one or more further activities can be performed in addition to those described. Still further, the order in which activities are listed are not necessarily the order in which they are performed.

The preferred embodiment of the invention, a distal femur replacement, consists of a distal femur body 100 rigidly attached to a cylinder 200 which houses a piston 250 such that piston 250 is capable of telescoping into and out of cylinder 200. A first pin 205 is pressed into a hole 201 in cylinder 200 and slides in a slot 256 in piston 250 in order to capture piston 250 within cylinder 200, prevent rotation between cylinder 200 and piston 250 and limit the amount of translation between the two parts. A thrust bearing 300 is attached to a threaded shaft 220 by sliding thrust bearing 300 over a shank 224 of threaded shaft 220 and placing a retaining ring 290 into a retaining ring groove 221. Alternatively, a thrust bushing may be used instead of a thrust bearing. Threaded shaft 220 is captured within distal femoral body 100 when cylinder 200 is rigidly attached to distal femur body 100. The male right-hand threads 222 of threaded shaft 220 engage female right-hand threads 252 in piston 250 so that counterclockwise rotation of threaded shaft 220 will translate piston 250 out of cylinder 200, effectively lengthening the device. Alternatively, left-hand threads could be used so that clockwise rotation of threaded shaft 220 would lengthen the device.

A femoral attachment adapter 350 incorporating a femoral stem 355, a female taper 352, and an alignment pin 354, is connected to piston 250 by pressing a male taper 252 into female taper 352 while aligning an alignment slot 254 to capture alignment pin 354. A tibial component 500, incorporating a tibial stem 505, a pair of knuckles 502, and a bumper 520, is connected to distal femur body 100 by a simple hinge. Bumper 520, preferably made from polyethylene, prevents wear between distal femur body 100 and tibial component 500 when fully extending the knee. To further prevent wear, the preferred embodiment of the invention incorporates a polyethylene bushing 450 between distal femur body 100 and the three-piece axle and polyethylene bushings between knuckles 502 and the three-piece axle. Tibial component 500 is supported by a tibial bearing 600 and is allowed to rotate around the axis of tibial stem 505 relative to tibial bearing 600. This rotation is limited by a pin 700 projecting from the underside of tibial component 500 which travels in an arcuate slot 602 in tibial bearing 600.

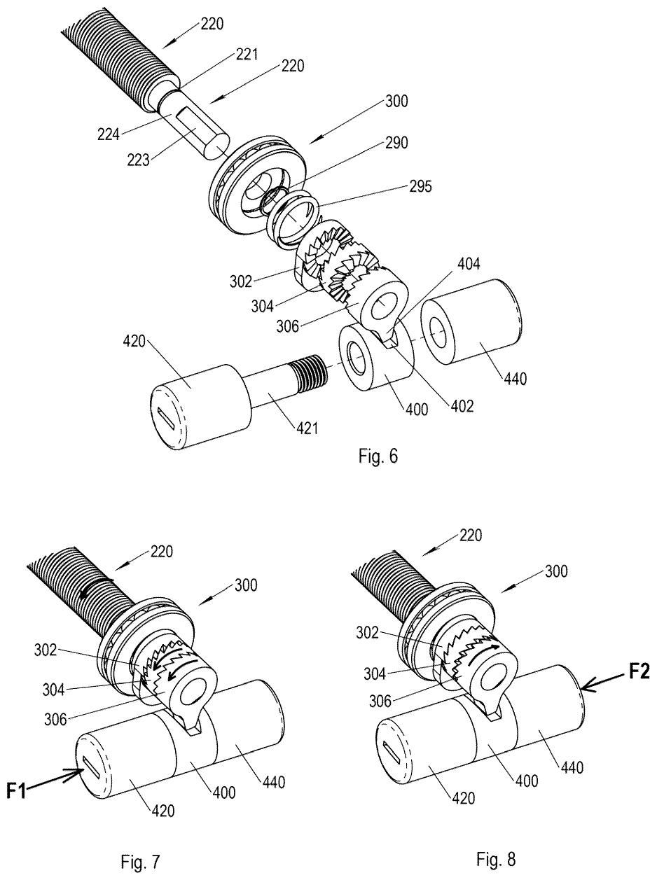

FIGS. 6, 7 and 8 show the action of the lengthening mechanism in greater detail by isolating it from the rest of the device. The lengthening mechanism consists of threaded shaft 220, retaining ring 290, a spring 295, thrust bearing 300, an idler ratchet 302, a drive ratchet 304, a pinion ratchet 306 and a three-piece axle consisting of an axle bolt 420 with a threaded shank 421, an axle nut 440 with female threads, and an axle rack 400 with a through hole 401. The goal of the lengthening mechanism is to rotate threaded shaft 220 counterclockwise (in the preferred embodiment) to push piston 250 out of cylinder 200 in order to lengthen the device. Idler ratchet 302 has a round hole through its center to accommodate shank 224, and has an oblong shape which fits into a similarly shaped pocket created in distal femur body 100. Therefore, idler ratchet 302 will not rotate relative to distal femur body 100 but is able to translate along and rotate about shank 224.

Drive ratchet 304 has a hole through its center which is D-shaped to communicate with a flat 223 on shank 224, thereby preventing relative rotation between shank 224 and drive ratchet 304, but allowing drive ratchet 304 to translate along shank 224. Rotation of drive ratchet 304 will therefore rotate threaded shaft 220. Alternatively, shank 224 could be a keyed shaft or a splined shaft and drive ratchet 304 could contain a keyway or splined hole accordingly to achieve the same action between threaded shaft 220 and drive ratchet 304. Pinion ratchet 306 has a round hole and is able to both translate along and rotate about shank 224. A compression spring 295 maintains tooth engagement between the three ratchets. Pinion ratchet 306 is situated at the bottom of a blind recess so that it cannot translate off of shank 224 once the device is assembled. Axle rack 400 incorporates a slot 402 which communicates with a cam 404 in pinion ratchet 306.

The interaction of axle rack 400 and pinion ratchet 306 is that of a rack and pinion: as axle rack 400 is translated to the right and to the left (as shown in the figures) it rotates pinion ratchet 306 counterclockwise and clockwise respectively. The rear face of pinion ratchet 306 incorporates sawtooth shaped teeth which communicate with sawtooth shaped teeth on the front face of drive ratchet 304. When the three-piece axle is pushed with force F1, thereby rotating pinion ratchet 306 counterclockwise (FIG. 7), these teeth lock together and pinion ratchet 306 drives the drive ratchet 304 and threaded shaft 220 counterclockwise. However, when the three-piece axle is pushed with force F2, thereby rotating pinion ratchet 306 clockwise (FIG. 8), the teeth freewheel and drive ratchet 304 is not driven. Essentially, pinion ratchet 306 and drive ratchet 304 form a one-way clutch. The sawtooth shaped teeth on the rear face of drive ratchet 304 and the front face of idler ratchet 302, which are oppositely configured from the teeth between pinion ratchet 306 and drive ratchet 304, will freewheel when drive ratchet 304 is rotated counterclockwise, but will otherwise lock together.

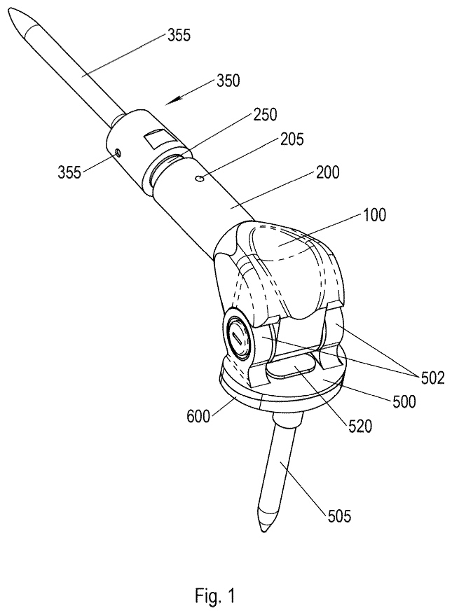

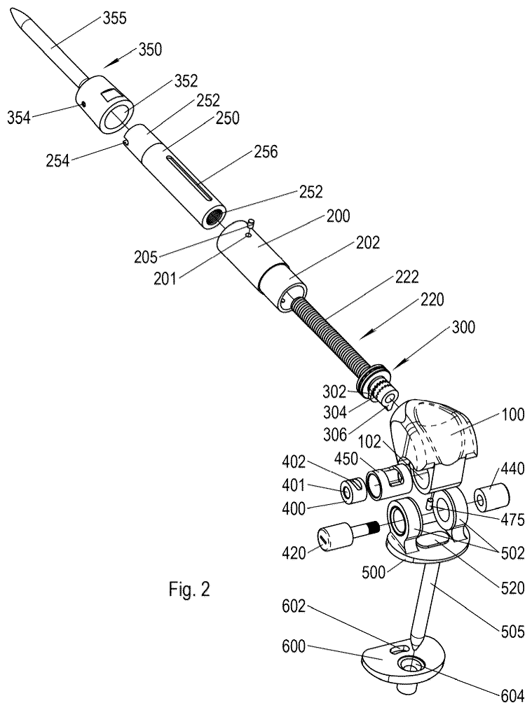

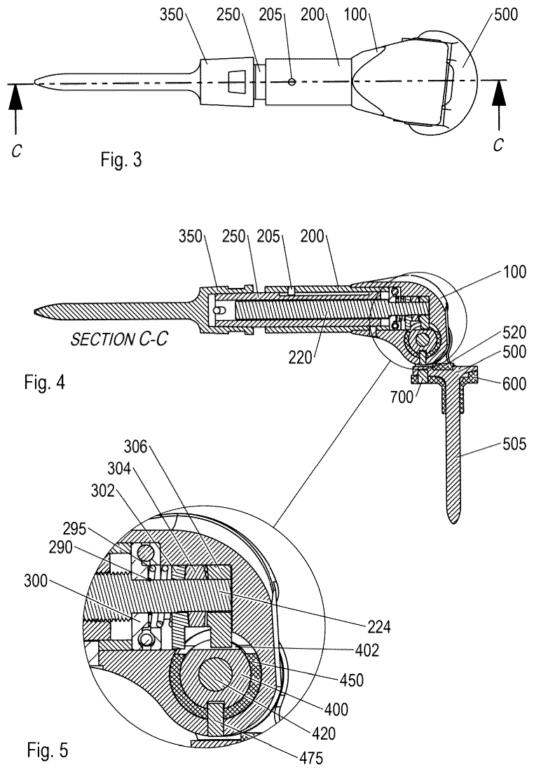

Essentially, the interaction of drive ratchet 304 and idler ratchet 302 is to prevent drive ratchet 304 from ever rotating clockwise. The force of spring 295 creates an audible “click” whenever a freewheel occurs between teeth. So, when axle rack 400 is fully translated to the right under the action of force F1 a “click” occurs between drive ratchet 304 and idler ratchet 302, and when axle rack 400 is fully translated to the left under the action of force F2, a “click” occurs between pinion ratchet 306 and drive ratchet 304. The “clicks” alert the operator that the axle has been pushed far enough to lengthen the device. Importantly, less than a full translation of axle rack 400 will not “click” and will not lengthen the device, so inadvertent small forces will not unintentionally lengthen the device. FIG. 1 demonstrates that the three-piece axle forms a recessed button to further protect against inadvertent lengthening due to the fact that lengthening forces must be exerted only on the three-piece axle in order to press it into the knuckle 502.

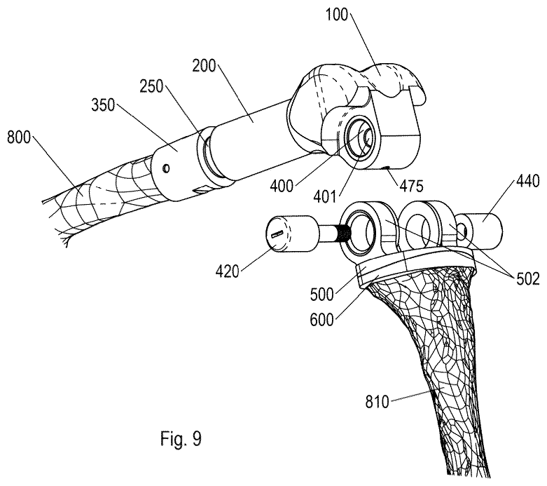

FIG. 9 demonstrates the assembly of the three-piece axle. Axle rack 400 is captured within distal femoral body 100 by pressing a second pin 475 into a hole in distal femoral body 100 and into a slot in axle rack 400 (not shown) so that axle rack 400 can translate right and left an amount determined by the length of said slot. Ideally, the length of said slot is set so that full travel of axle rack 400 creates one ratchet tooth advance, or “click”, in each direction. The surgical procedure includes resection of the distal aspect of a femur 800 and creation of a round hole to accommodate femoral stem 355. The proximal aspect of a tibia 810 is prepared for attachment of tibial bearing 600 and a round hole is created to accommodate tibial stem 505. The femoral portion of the device is then attached to femur 800 and the tibial portion of the device is attached to tibia 810, as shown in FIG. 9. Distal femoral body 100 is then inserted into the slot created by knuckles 502 in tibial component 500 so that hole 401 in axle rack 400 is aligned with holes in knuckles 502. Then, axle bolt 420 is passed through hole 401 and captured by axle nut 440 which threads fully onto axle bolt 420.

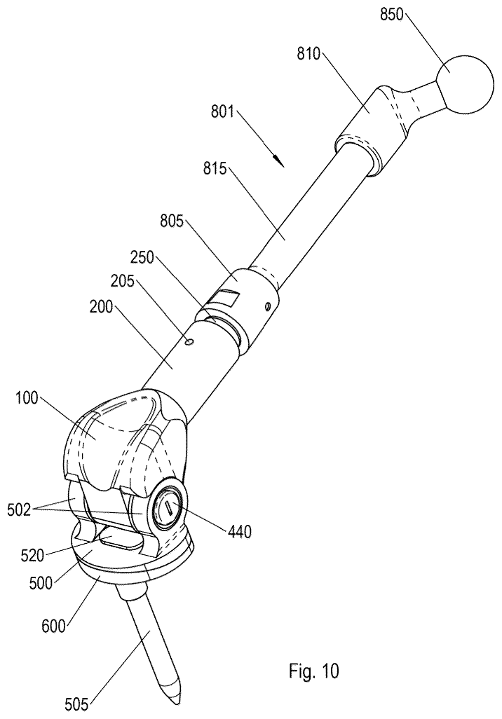

A total femur replacement, where the distal femur replacement described above is attached to a proximal femur body 801 instead of femoral attachment adapter 350, is depicted in FIG. 10. Proximal femur body 801 incorporates an adapter 805 with a female taper similar to female taper 352. A shaft 815, a proximal body 810, and a femoral ball 850 are also incorporated. Femoral ball 850 would typically communicate with either the patient's acetabulum or an acetabular cup affixed to the patient's pelvis. The lengthening procedure for the total femur replacement would be identical to that of the previously described distal femur replacement.

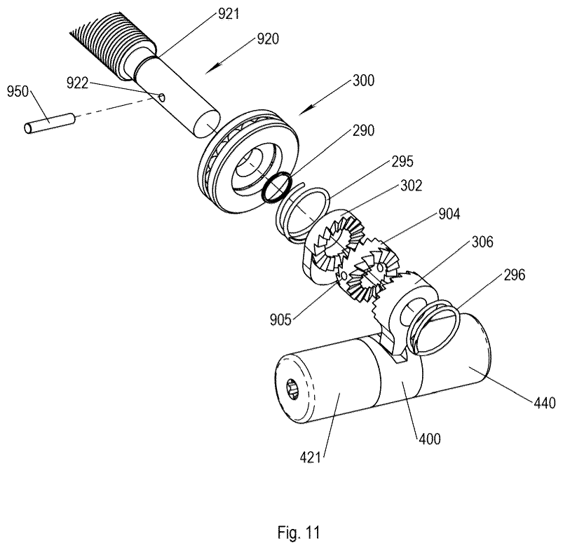

A second alternative embodiment of the invention is shown in FIG. 11. Here, an alternative drive ratchet 904 is rigidly attached to an alternative threaded shaft 920 with a dowel pin 950 pressed into hole 905 and hole 922, so that it will not rotate or translate relative to threaded rod 920. Other methods of rigid attachment, such as welding, adhesive, press-fit, or cold weld could be utilized. Both pinion ratchet 306 and idler ratchet 302 are able to rotate and translate along alternative threaded shaft 920. Spring 295 pushes idler ratchet 302 such that the teeth engage with the communicating teeth in alternative drive ratchet 904. Similarly, a second spring 296 pushes pinion ratchet 306 such that the teeth engage with the communicating teeth in alternative drive ratchet 904. When pinion ratchet 306 is rotated counterclockwise it rotates alternative drive ratchet 904 and idler ratchet 302 freewheels by translating away from alternative drive ratchet 904. When pinion ratchet 306 is rotated clockwise it freewheels by translating away from alternative drive ratchet 904 while the teeth on idler ratchet 302 maintain contact with the teeth on alternative drive ratchet 904 preventing rotation.

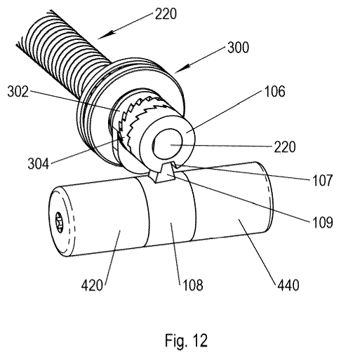

Another alternative embodiment of the invention is shown in FIG. 12 wherein a slot 107 in alternative pinion ratchet 106 communicates with a cam 109 on alternative axle rack 108. This is the reverse of the preferred embodiment, but will have similar action.

All references cited herein are expressly incorporated by reference in their entirety. It will be appreciated by persons skilled in the art that the invention is not limited to what has been particularly shown and described herein above. In addition, unless mention was made above to the contrary, it should be noted that all of the accompanying drawings are not to scale. There are many different features to the invention and it is contemplated that these features may be used together or separately. Thus, the invention should not be limited to any particular combination of features or to a particular application of the invention. Further, it should be understood that variations and modifications within the spirit and scope of the invention might occur to those skilled in the art to which the invention pertains. Accordingly, all expedient modifications readily attainable by one versed in the art from the invention set forth herein that are within the scope and spirit of the present invention are to be included as further embodiments of the invention.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended claims or claim elements unless the exact words “means for” or “step for” are explicitly used in the particular claim, followed by a participle phrase identifying a function.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the claims.

After reading the disclosure, skilled artisans will appreciate that certain features are, for clarity, described herein in the context of separate embodiments, can also be provided in combination in a single embodiment. Conversely, various features that are, for brevity, described in the context of a single embodiment, can also be provided separately or in any sub-combination. Further, references to values stated in ranges include each and every value within that range.

The above discussion is meant to be illustrative of the principles and various embodiments of the present invention. Numerous variations and modifications will become apparent to those skilled in the art once the above disclosure is fully appreciated.

What is claimed is:

1. A non-invasively extendable endoprosthesis incorporating:
a first feature configured for attachment to a first long bone;
a second feature configured for attachment to a second, adjacent long bone;
a joint having at least one rotational degree of freedom which is intended to replace the function of an orthopedic joint; and
a lengthening mechanism which is purely mechanical and manually operates while being located and activated by an operator post-operatively by pressing on skin of the patient, without requiring a surgical incision, a magnet, a hydraulic or pneumatic component, electrical current or application of heat, the lengthening mechanism having
a threaded shaft rotatably connected to the first feature to lengthen or shorten the first feature when the threaded shaft is rotated;
a drive ratchet engaged with the threaded shaft and having first teeth on a first side of the drive ratchet;
a pinion ratchet having teeth which are engageable with the first teeth of the drive ratchet to cause rotation of the drive ratchet when the pinion ratchet is rotated in a first direction to thereby cause rotation of the threaded shaft, the teeth of the pinion ratchet disengagable from the first teeth of the drive ratchet when the pinion ratchet is rotated in a second direction opposite to the first direction; and
a frame affixable to the body and supporting the drive ratchet and the pinion ratchet.

2. The endoprosthesis of claim 1, wherein the pinion ratchet and drive ratchet are mutually separable by translation to enable the pinion ratchet to disengage from the first teeth of the drive ratchet when the pinion ratchet is rotated in the second direction.

3. The endoprosthesis of claim 2, wherein the pinion ratchet rotates axially about the longitudinal axis of the threaded shaft.

4. The endoprosthesis of claim 1, wherein the drive ratchet includes second teeth on a side of the drive ratchet opposite to the first teeth, the endoprosthesis further including
an idler ratchet translatable along the longitudinal axis of the threaded shaft and connected to the frame to limit radial rotation of the idler ratchet about the longitudinal axis of the threaded shaft,
the threaded shaft rotatable within a bore of the idler ratchet,
teeth of the idler ratchet engageable with the second teeth of the drive ratchet to prevent rotation of the drive ratchet and thereby the threaded shaft when the drive ratchet is not being rotated in the first direction by the pinion ratchet,
the idler ratchet and the drive ratchet mutually separable from engagement by translation when the pinion ratchet is rotated in the first direction, and
the drive ratchet being translatable along the longitudinal axis of the threaded shaft, and limited in radial rotation about the longitudinal axis of the threaded shaft.

5. The endoprosthesis of claim 4, further including a biasing element positioned between the frame and the idler ratchet to bias the idler ratchet, drive ratchet, and pinion ratchet into mutual engagement.

6. The endoprosthesis of claim 4, the threaded shaft having an axially extending engagement region that is slidably engageable with a mating region within the bore of the drive ratchet to thereby prevent the radial rotation of the drive ratchet relative to the threaded shaft, while enabling translation of the drive ratchet along the longitudinal axis of the threaded shaft.

7. The endoprosthesis of claim 2, further including an axle, the axle including a slot, the pinion ratchet including a cam, the cam of the pinion ratchet engaged within the slot, whereby movement of the axle causes relative movement of the cam causing rotation of the pinion ratchet in the first or second direction, an end of the axle pressable by the pressing on skin of the patient.

8. The endoprosthesis of claim 7, the axle disposed at an angle relative to the longitudinal axis of the threaded shaft.

9. The endoprosthesis of claim 1, wherein the frame forms a part of the joint.

10. The endoprosthesis of claim 9, wherein the axle forms a rotational axis of the joint.

11. An endoprosthesis extendable in length without cutting skin of the patient, the endoprosthesis comprising:
a proximal end attachable to an object forming a portion of an elongate bone of the patient;
a threaded shaft rotatably attached to the proximal end;
a distal end connected to the threaded shaft, whereby rotation of the threaded shaft causes relative separation of the proximal and distal ends thereby lengthening the elongate bone of the patient;
a drive ratchet engaged with the threaded shaft and having first teeth on a first side of the drive ratchet;
a pinion ratchet having teeth which are engageable with the first teeth of the drive ratchet to cause rotation of the drive ratchet when the pinion ratchet is rotated in a first direction to thereby cause rotation of the threaded shaft, the teeth of the pinion ratchet disengagable from the first teeth of the drive ratchet when the pinion ratchet is rotated in a second direction opposite to the first direction, the pinion rachet rotatable by pressing on skin of the patient; and
a frame affixable to the body and supporting the drive ratchet and the pinion ratchet;
the length of the endoprosthesis increasable by rotating the pinion ratchet by the pressing of the skin, and without a requirement of forming a surgical incision, and without using a magnet, hydraulic or pneumatic component, electrical current, or an application of heat.

12. The endoprosthesis of claim 11, the frame forming a portion of a joint replacement.

13. The endoprosthesis of claim 11, wherein the pinion ratchet and drive ratchet are mutually separable by translation to enable the pinion ratchet to disengage from the first teeth of the drive ratchet when the pinion ratchet is rotated in the second direction.

14. The endoprosthesis of claim 11, wherein the pinion ratchet rotates radially about the longitudinal axis of the threaded shaft.

15. The endoprosthesis of claim 11, wherein the drive ratchet includes second teeth on a side of the drive ratchet opposite to the first teeth, the endoprosthesis further including
an idler ratchet translatable along the longitudinal axis of the threaded shaft and connected to the frame to limit radial rotation of the idler ratchet about the longitudinal axis of the threaded shaft,
the threaded shaft rotatable within a bore of the idler ratchet,
teeth of the idler ratchet engageable with the second teeth of the drive ratchet to prevent rotation of the drive ratchet and the threaded shaft when the drive ratchet is not being rotated in the first direction by the pinion ratchet,
the drive ratchet being limited in radial rotation about the longitudinal axis of the threaded shaft, and
the idler ratchet translatable away from engagement with the drive ratchet when the pinion ratchet is rotated in the first direction.

16. The endoprosthesis of claim 15, further including a biasing element positioned between the frame and the idler ratchet to bias the idler ratchet into engagement with the drive ratchet.

17. The endoprosthesis of claim 14, the threaded shaft having an axially extending engagement region that is engageable with a mating region within the bore of the drive ratchet to thereby prevent the radial rotation of the drive ratchet about the elongated axis of the threaded shaft.

18. The endoprosthesis of claim 12, further including an axle, the axle and the pinion ratchet mutually engaged by a cam on one of the axle and pinion ratchet and a slot on the other of the axle and pinion ratchet, whereby movement of the axle causes relative movement of the cam and slot causing rotation of the pinion ratchet in the first or second direction, an end of the axle pressable by the pressing on skin of the patient.

19. The endoprosthesis of claim 9, wherein the axle forms a rotational axis of the joint.

20. An endoprosthesis extendable in length without cutting skin of the patient, the endoprosthesis comprising:
a proximal end attachable to an object forming a portion of an elongate bone of the patient;
a threaded shaft rotatably attached to the proximal end;
a distal end connected to the threaded shaft, whereby rotation of the threaded shaft causes relative separation of the proximal and distal ends thereby lengthening the elongate bone of the patient;
a drive ratchet engaged with the threaded shaft and limited in radial rotation about the threaded shaft and having first teeth on a first side of the drive ratchet and second teeth on a second side opposite to the first side;
a pinion ratchet having teeth which are engageable with the first teeth of the drive ratchet to cause rotation of the drive ratchet when the pinion ratchet is rotated in a first direction to thereby cause rotation of the threaded shaft, the teeth of the pinion ratchet disengagable from the first teeth of the drive ratchet by translation of the pinion ratchet relative to the drive ratchet, when the pinion ratchet is rotated in a second direction opposite to the first direction, the pinion rachet rotatable by pressing on skin of the patient;
an idler ratchet connected to the frame to limit radial rotation of the idler ratchet about the longitudinal axis of the threaded shaft;
the threaded shaft rotatable within a bore of the idler ratchet;
teeth of the idler ratchet engageable with the second teeth of the drive ratchet to prevent rotation of the drive ratchet and thereby the threaded shaft when the drive ratchet is not being rotated in the first direction by the pinion ratchet;
the drive ratchet being translatable relative to the drive pinion along the longitudinal axis of the threaded shaft to enable mutual engagement when the pinion ratchet is rotated in the first direction;
a frame affixable to the body and supporting the drive ratchet and the pinion ratchet; and
an axle, the axle and the pinion ratchet mutually engaged by a cam on one of the axle and pinion ratchet and a slot on the other of the axle and pinion ratchet, whereby movement of the axle causes relative movement of the cam and slot causing rotation of the pinion ratchet in the first or second direction, an end of the axle pressable by the pressing on skin of the patient;
the length of the endoprosthesis increasable by pushing the axle thereby rotating the pinion ratchet by the pressing of the skin, and without a requirement of forming a surgical incision, and without using a magnet, hydraulic or pneumatic component, electrical current, or an application of heat.
